(19) United States
(12) Patent Application Publication
(10) Pub. No.: US 2019/0350790 A1
(43) Pub. Date: Nov. 21, 2019

(54) PATIENT TABLE HAVING RECEIVING DEVICE FOR RECEIVING A TRANSFER BOARD ALONG TWO AXES

(71) Applicant: Siemens Healthcare GmbH, Erlangen (DE)

(72) Inventors: Matthias DROBNITZKY, Spardorf (DE); Kirsten LUDWIG, Schwarzenbruck (DE); Markus PETSCH, Erlangen (DE)

(73) Assignee: Siemens Healthcare GmbH, Erlangen (DE)

(21) Appl. No.: 16/404,951

(22) Filed: May 7, 2019

(30) Foreign Application Priority Data
May 16, 2018 (DE) 102018207639.1

(51) Int. Cl. A61G 7/10; A61B 5/055; A61B 6/04

(52) U.S. Cl. CPC A61G 7/103; A61G 2210/50; A61B 6/0407; A61B 5/0555

(57) ABSTRACT

A patient table of an imaging apparatus includes a receiving device for receiving a transfer device that is reversibly transferable to the patient table. In an embodiment, the receiving device is arranged on the patient table such that a surface region of the receiving device is in contact with the transfer device when the transfer device is received. Further, the receiving device is embodied to receive the transfer device reversibly along at least two axes that are different from one another in the horizontal plane.

### PRIORITY STATEMENT

The present application hereby claims priority under 35 U.S.C. § 119 to German patent application number DE 102018207639.1 filed May 16, 2018, the entire contents of which are hereby incorporated herein by reference.

### FIELD

Embodiments of the invention generally relate to a patient table of an imaging apparatus having a receiving device for receiving a transfer device that is reversibly transferable to the patient table along at least two axes that are different from one another in the horizontal plane.

### BACKGROUND

In the course of an operative treatment of a sedated or anesthetized patient it is often necessary to transfer the patient to an imaging apparatus, for example a magnetic resonance imaging apparatus, before, during and after the intervention in order to acquire corresponding images that are used to prepare for and monitor or control the treatment. This transfer frequently takes place via a movable patient support and positioning table that features a transfer board which is transferred from the patient support and positioning table to the patient table of the imaging apparatus.

An example of a treatment requiring image monitoring is a neurosurgical intervention. During the intervention, the patient lies on the transfer board, which is arranged on the neurosurgical operating table. In order to monitor the operation, the transfer board is now transferred from the operating table onto the patient support and positioning table, which is then moved to the patient table of the magnetic resonance system, where the transfer board is transferred once more. Following acquisition of the images, the transfer board is transferred back onto the patient support and positioning table.

Another example of a treatment is a catheter ablation, which is often carried out under X-ray control. During the ablation procedure, the transfer board is arranged on the table of a C-arm X-ray machine. The guiding of the catheter can be tracked in the course of the X-ray monitoring. In order to check on the success of the ablation, the transfer board is once again transferred onto the patient support and positioning table and from the latter to the patient table of the imaging apparatus, i.e. for example of the MR device, where the corresponding images are then recorded.

Alternatively, it is also possible to use a mobile patient table which can be decoupled from the imaging apparatus so that the transfer board can be transferred directly from an operating table to the patient table, and the patient table itself is used for transferring the patient.

In this case, in preparation for the transfer via transfer board, the patient table usually provides a device(s) for receiving the transfer board which permit the transfer board to be transferred, in contrast to a standard patient table which provides a substantially level surface for supporting and positioning the patient, on which the patient independently places him-/herself in a recumbent position.

### SUMMARY

At least one embodiment of the present invention provides a patient table of an imaging apparatus which permits a simplified transfer of a patient to the patient table in a manner that befits the situation, including in operative situations.

At least one embodiment of the inventive patient table of an imaging apparatus includes a receiving device. Advantageous embodiments which are inventive considered per se are the subject matter of the claims and the following description.

At least one embodiment of the invention relates to a patient table of an imaging apparatus having a receiving device for receiving a transfer device that is reversibly transferable to the patient table, the receiving device being arranged on the patient table in such a way that a contact surface area of the receiving device facing toward the transfer device is in contact with the transfer device when the transfer device is received, and the receiving device being embodied to receive the transfer device reversibly along at least two axes that are different from one another in the horizontal plane.

According to at least one embodiment of the invention, the receiving device is embodied to receive the transfer device along at least two axes that are different from one another in the horizontal plane and also to release the same again. In other words, the receiving device permits in particular the transfer device to be moved along at least two axes that are different from one another when it is being received. Preferably, at least the transfer of the transfer device along the longitudinal axis of the patient table and along the transverse axis of the patient table is facilitated by the receiving device. However, the receiving device may also permit the transfer along more than two axes.

At least one embodiment of the invention is directed to a magnetic resonance tomography imaging apparatus, comprising the patient table of at least one embodiment.

### BRIEF DESCRIPTION OF THE DRAWINGS

Example embodiments of the invention are explained in more detail below with reference to figures, in which:

FIG. 1 shows a schematic top plan view onto a patient table according to the invention in a first embodiment variant having a receiving device for receiving a transfer board along two axes that are different from one another,

FIG. 2 shows a schematic cross-section through an inventive patient table according to the first embodiment variant,

FIG. 3 shows a schematic top plan view onto a patient table according to the invention in a second embodiment variant having a receiving device for receiving a transfer device along two axes that are different from one another,

FIG. 4 shows a schematic top plan view onto a patient table according to the invention in a third embodiment variant having a receiving device for receiving a transfer board along two axes that are different from one another.

### DETAILED DESCRIPTION OF THE EXAMPLE EMBODIMENTS

In the following, embodiments of the invention are described in detail with reference to the accompanying drawings. It is to be understood that the following description of the embodiments is given only for the purpose of illustration and is not to be taken in a limiting sense. It should be noted that the drawings are to be regarded as being schematic representations only, and elements in the drawings are not necessarily to scale with each other. Rather, the representation of the various elements is chosen such that their function and general purpose become apparent to a person skilled in the art.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a patient table of an imaging apparatus having a receiving device for receiving a transfer device that is reversibly transferable to the patient table, the receiving device being arranged on the patient table in such a way that a contact surface area of the receiving device facing toward the transfer device is in contact with the transfer device when the transfer device is received, and the receiving device being embodied to receive the transfer device reversibly along at least two axes that are different from one another in the horizontal plane.

According to at least one embodiment of the invention, it has been recognized that in the case of patient tables which enable a patient to be transferred with the aid of a transfer device, i.e. for example which permit a transfer board to be received, the receiving direction is usually limited to one axis, in most cases along the longitudinal axis of the patient table. Alternatively, methods that are complex in terms of their construction, for example making use of an air cushion, find application in order to enable a transfer of a patient onto and from the patient table both along and transversely to the longitudinal axis of the patient table. However, the ability to receive the transfer device along different axes, at least along the longitudinal and the transverse axis of the patient table, permits a patient to be transferred to the patient table of the imaging apparatus as requirements dictate and as befits the particular situation, and allows a simplified and time-efficient workflow during a treatment or therapy of a patient, in particular in operative situations.

At least one embodiment of the invention advantageously permits the use of simple device(s) of construction to enable a reversible reception of a transfer device along different axes in the horizontal plane. A transfer of a patient from a patient support and positioning table or an operating table to the patient table of the imaging apparatus is thus made possible in accordance with requirements and as befits the particular situation.

What is meant by the reversible reception of a transfer device by the patient table in this context is that the transfer device can be transferred for example from a further patient support and positioning table to the patient table of the imaging apparatus and can also be transferred back again. In this case the transfer device can be moved manually or with the aid of a motor. For example, the transfer device can slide as a result of a pushing or pulling action using physical strength in particular in a horizontal plane from a patient support and positioning table onto the patient table.

The transfer device may be equivalent to a transfer board with or without a patient self-supporting function or alternatively, in the simplest case, to a transfer sheet on which a patient is placed in order to be able to be transferred between different patient support and positioning tables, i.e. the transfer device can be embodied at least in such a way that it can be displaced for example in the horizontal plane with the patient lying thereon.

The receiving device is arranged in this case on the patient table in such a way that a contact surface area is available which, while the transfer device is moved when being received in the horizontal plane, is in contact with the transfer device and faces toward the transfer device, i.e. the support and positioning plane of the patient. For example, the receiving device is seated on a substantially horizontal section of the patient table and has, opposite the overlying side, a side whose surface is embodied in part or wholly as a contact surface area for the transfer device.

The contact surface area may in this case comprise a continuous surface or a plurality of point-by-point and/or discontinuous surfaces. The contact surface area may in this case be embodied in particular in such a way that it is possible for a movement of the transfer device over the contact surface area to be performed with little resistance. The contact surface area can be embodied as smooth and having low frictional resistance, for example, such that the transfer device can be slidably guided and moved over the contact surface area.

The receiving device may, for example, also comprise casters or ball casters which are in contact with the transfer device, in particular a transfer board, and which, when the transfer device is displaced due to the rolling action of the casters along the movement direction, permit the transfer device to be moved with little resistance. The casters are mounted via a sliding bearing, for example.

The receiving device may comprise for example a fiber-reinforced composite material, a plastic material or a metal, or a combination thereof. Preferably, the guideway comprises a material which is nonconducting and nonmagnetic, for example a fiber-reinforced composite material or a plastic material.

According to at least one embodiment of the invention, the receiving device is embodied to receive the transfer device along at least two axes that are different from one another in the horizontal plane and also to release the same again. In other words, the receiving device permits in particular the transfer device to be moved along at least two axes that are different from one another when it is being received. Preferably, at least the transfer of the transfer device along the longitudinal axis of the patient table and along the transverse axis of the patient table is facilitated by the receiving device. However, the receiving device may also permit the transfer along more than two axes.

The receiving device can in this case bear on the patient table or simply have sections which bear on the patient table and are in contact with the patient table. In particular, the receiving device and the patient table may be embodied in such a way that between the receiving device and the patient table there is available a receiving space which permits optional elements to be arranged between the receiving device and the patient table. For example, the receiving space may be embodied in such a way that an optional functional imaging element, for example a local coil in magnetic resonance tomography, can be introduced into the receiving space and consequently can also be placed beneath a patient or, even when the functional imaging element is used, the patient can be transferred on a transfer device in a horizontal plane and along at least two axes different from one another from a patient support and positioning table onto the patient table.

The receiving device can in this case be wholly or partially releasably connectable to the patient table, i.e. with simple or a without third device, for example purely manually and using physical strength, can be repeatedly arranged on the patient table and nondestructively released again. Advantageously, the patient table can easily be adapted to suit imaging conditions and therapy or treatment situations. Advantageously, a versatile use of the patient table is possible. Alternatively, however, the receiving device can also be permanently connected to the patient table.

The imaging apparatus can be chosen for example from the group comprising a computed tomography device, a PET-CT device, a SPECT-CT device, a SPECT device, an X-ray machine, a C-arm X-ray machine, a magnetic resonance tomography device, and combinations thereof.

Advantageously, the receiving device permits a transfer device to be received along at least two axes, at least the longitudinal and the transverse axis, using simple device(s). Advantageously, the patient table together with the receiving device enables a patient to be transferred as required to the patient table of the imaging apparatus and allows a simplified workflow during a treatment of a patient.

In an advantageous embodiment of the invention, it is provided that the receiving device comprises a sliding board and the patient table includes a recess into which the sliding board can be inserted. In this case, with sliding board inserted, the surface of the sliding board fits flush with a surface region of the patient table forming a receiving surface for the transfer device in combination with the surface of the sliding board.

The sliding board can have a smooth surface facing toward the transfer device and having a low frictional resistance so that the transfer device, for example a transfer sheet or a transfer board, can be slidably moved on its surface against minimal resistance. For example, the sliding board has a lower frictional resistance on the surface facing toward the transfer device than on the side facing toward the patient table and facing away from the transfer device. A transfer board having casters on the side facing toward the sliding board can also be used, for example. Advantageously, an easy and time-efficient transfer is possible. Advantageously, a sliding board represents a simple and cost-effective ways of enabling a patient to be received along different axes.

The patient table makes available in particular a recess which is adapted to fit the sliding board and into which the sliding board can be inserted. In this arrangement, the sliding board can be insertable reversibly and without third devices or tools and be extractable again from the recess. For example, the sliding board can have a handle by which the sliding board can be raised and which is embodied to enable an easy insertion and removal of the sliding board. With the sliding board inserted into the recess, the surface of the sliding board forms a receiving surface for the transfer device in combination with the surface region of the patient table which is not covered by the sliding board and adjoins the recess and which faces toward the support and positioning plane of the patient, for example housing parts.

By way of the receiving surface, the transfer device can be transferred onto the patient table via different axes. The transfer device can be moved along different axes on the receiving surface. In this case the surface of the sliding board fits flush with the surface region of the patient table, i.e. without vertical offset or steps obstructing the reception of the transfer device between the sliding board and the surface region of the patient table, such that it is easily possible to receive the sliding board in a horizontal plane. The sliding board can for example comprise a fiber-reinforced composite material, a plastic material or a metal, or a combination thereof. Preferably, the sliding board includes a material which is nonconducting and nonmagnetic, for example a fiber-reinforced composite material or a plastic material.

Advantageously, a sliding board inserted into a recess of the patient table enables a transfer device to be transferred along different axes. Advantageously, different transfer device can be used, including such without a self-supporting function for the patient. Advantageously, a sliding board represents a receiving device for a transfer device that is simple to produce, low in cost and easy to handle.

In a further advantageous embodiment of the invention, the patient table and the sliding board are embodied in such a way that a receiving space for accommodating a functional imaging element is embodied between the inserted sliding board and the patient table.

Functional imaging elements may for example comprise optional local coils used in magnetic resonance tomography, which are placed underneath the patient on the patient table for the purpose of an examination in the imaging apparatus. The local coil is a spine coil, for example. However, functional imaging elements may also comprise additional electronics or signal lines, for example.

The patient table and the sliding board can in this case be embodied in such a way that the functional imaging element is placed on the patient table before the sliding board is inserted or, alternatively, if for example the sliding board is already inserted, can also be slipped under the sliding board from the side.

Advantageously, the functionality of the patient table can be adapted to suit the examination. Advantageously, the sliding board enables a patient to be received horizontally, for example from another patient support and positioning table, in spite of functional imaging elements arranged on the patient table which can make a horizontal transfer more difficult due to points of unevenness occurring and vertical offset surface areas. Advantageously, easy placement of the sliding board under a patient is possible.

In a further advantageous embodiment of the invention, the sliding board furthermore has at least one depression which is embodied to catch fluid.

The fluids can include body fluids which are collected by the depression. The sliding board is preferably embodied in such a way that fluids collect in the depression, i.e. the surface can be embodied for example with a slight incline running down toward the depression.

Advantageously, this enables other elements arranged on and/or around the patient table to be protected against fluid. Easier cleaning is advantageously possible.

In an advantageous embodiment of the invention, the receiving device has a plurality of ball casters in a horizontal plane which, when a transfer device is received, are in contact with the transfer device. In this embodiment, the transfer device is limited to a transfer board which possesses a sufficiently high degree of rigidity to allow a displacement even when there is only a pointwise support and contact area between the receiving device and the transfer board, i.e. between the ball casters and the transfer board. The ball casters embodied in a horizontal plane have no distinct axis of rotation and consequently have no preferred orientation in respect of the movement of a transfer board when the transfer board is shifted on the ball casters. The ball casters can support a constant and uniform movement of the transfer board on the patient table by virtue of a rolling movement of the casters when the transfer board is displaced, for example by pushing or pulling of the transfer board, in the direction of the rolling movement. As a result, a gentle, shock- and vibration-free accommodation of the transfer board and transfer of the patient is advantageously possible. Moreover, an easy and low-friction guidance of the transfer board can be achieved as a result.

In this embodiment, the receiving device furthermore has a first rail system along a first axis and a second rail system along a second axis that is different from the first axis, the first rail system guiding the transfer board when the transfer board is received along the first axis and the second rail system guiding the transfer board when the transfer board is received along the second axis. The first and/or the second rail system can comprise a single rail or a plurality of rails. Preferably, the first rail system is aligned along the longitudinal axis and the second rail system is aligned along the transverse axis of the patient table, and they guide the transfer board along the longitudinal axis and along the transverse axis of the patient table, respectively.

The transfer board can then have a corresponding first guide element or a plurality of first guide elements which interact with the first rail system when the transfer board is received along the longitudinal axis. The interaction can be embodied in such a way that the transfer board is able to perform a secure and reliable movement along the longitudinal axis, and a movement along another axis that is different from the longitudinal axis is prevented. The transfer board can have a corresponding second guide element or a plurality of second guide elements which interact with the second rail system when the transfer board is received along the transverse axis, such that the transfer board performs a secure and reliable movement along the transverse axis.

In the simplest case, the first or second rail system can comprise a groove or a plurality of grooves along the longitudinal axis or, as the case may be, a groove or a plurality of grooves along the transverse axis in the material of the receiving device or of the patient table, into which groove or grooves the first or the second guide element or the plurality of first and the plurality of second guide elements of the transfer board can be introduced.

A secure and reliable reception of the transfer board along the longitudinal axis and along the transverse axis of the patient table is advantageously possible.

The receiving device can in this case comprise a level board on which the ball casters are embodied and which can be placed on the patient table. The receiving device can also comprise a plurality of connected components on which the ball casters are embodied. The receiving device can also comprise a plurality of unconnected components on which the ball casters are embodied and which can for example be placed individually on the patient table. The receiving device can be nondestructively connected in a releasable manner to the patient table and be repeatedly detached from the patient table and reattached thereto. A versatile use of the patient table is possible as a result. For example, the receiving device can be placed onto the patient table or inserted into a recess of the patient table.

In an advantageous embodiment of the invention, the receiving device has a first number of guideways for guiding a transfer board along a first axis and in addition a second number of guideways for guiding the transfer board along a second axis different from the first axis. In particular, only a discontinuous contact surface area for the transfer device is provided by way of the guideways, such that the transfer device in this embodiment of the invention is limited to one transfer board, i.e. a rigid device embodied in a planar shape.

In this way it can be ensured that the transfer board can be moved horizontally along the first or second axis even if the transfer board is only in contact in sections with the receiving device or the patient table and a patient can be supported and positioned on the patient table for the purpose of an examination. In this case the receiving device additionally comprises a device for adjusting the relative height between the first number of guideways and the second number of guideways, such that there is a first setting of the relative height in which the transfer board is guided by the first number of guideways and there is a second setting of the relative height in which the transfer board is guided by the second number of guideways. In this case the first and the second number of guideways can comprise only one guideway or a plurality of guideways, the number, embodiment and arrangement of the guideway or guideways being chosen so as to enable a stable guiding of the transfer board when it is being received.

A guideway can be in particular a workpiece which, by virtue of the embodiment of the side facing toward the transfer device and the support and positioning plane of the patient, presents a linear preferred orientation for the movement of the transfer device, which is guided along the guideway while being received on or transferred back from the patient table. In this embodiment of the invention, the receiving device can therefore have a first number of guideways along a first axis which present a preferred orientation for the movement of the transfer device along the first axis and a second number of guideways along a second axis which present a preferred orientation for the movement of the transfer device along the second axis. Along the preferred orientation for the movement, the transfer device can be moved in particular in both directions, i.e. forwards and backwards.

The guideways of the first and/or second number of guideways present for example a smooth rail along which the transfer board can be slidably moved as soon as it comes into contact with the first or with the second number of guideways. The guideways also comprise for example casters having a designated rotational axis such that the transfer board is in contact with the casters and, when the transfer board is displaced perpendicularly to the rotational axis, the rolling movement of the casters enables the transfer board to be guided perpendicularly to the rotational axis. However, the guideways may also be embodied in some other way. Preferably, the guideways are embodied in such a way that the transfer board can be moved substantially horizontally on the patient table. Preferably, a uniform and constant movement of the transfer board can be made possible as soon as it comes into contact with the first or with the second number of guideways. This enables a gentle, shock- and vibration-free reception of the transfer board and the patient to be ensured.

According to at least one embodiment of the invention, the relative height between the first and the second number of guideways can be set by a device for adjusting the relative height, such that there is a first setting in which the first number of guideways are in contact with the transfer board and, while being received, the transfer board can be guided along their preferred orientation, and such that there is also a second setting of the relative height in which the second number of guideways are in contact with the transfer board and guide the transfer board along their preferred orientation. There can also be further settings in addition to the first and second setting. In this case a portion of the first or second number of guideways can be fixed relative to the patient table and the other portion of the number can be embodied as adjustable in height relative thereto, or else both the first and the second number of guideways are embodied as adjustable in height relative to the patient table.

A guideway of the first and the second number of guideways can be produced from one component or alternatively can be assembled from multiple components. Guideways of the first and/or the second number of guideways can also be interconnected. The guideways comprise for example a fiber-reinforced composite material, a plastic material or a metal, or a combination thereof. Preferably, the guideway contains a material which is nonconducting and nonmagnetic, for example a fiber-reinforced composite material or a plastic material.

The guideways can also bear on the patient table or for example adjoin laterally to a side part or to a housing part of the patient table and thus be adapted to match the design of the patient table. This enables a stable arrangement of the guideway on the patient table to be realized.

The device for adjusting the relative height can comprise for example a mechanical device with a manually operable handle which, when the handle is turned or lowered or raised, leads via a mechanical transmission to a setting of the relative height between the first and the second number of guideways. The handle is part of a lever device, for example. In this way, for example, a nonmagnetic and nonconducting device for adjusting the relative height can be realized. Other example embodiments can also include electronic components and a motor-driven or hydraulic device for adjusting the relative height.

The device for adjusting the relative height can be arranged on the receiving device, for example on the side or in an area of the patient table and the receiving device that is not covered by the transfer board and made inaccessible by the transfer board, such that the relative height is also adjustable when a transfer board has been received, so that for example a transfer board can be received along the longitudinal axis of the patient table and transferred back again via the transverse axis. However, the device for adjusting the relative height can also be arranged on the receiving device in such a way that the adjustment is possible only at a time prior to a reception of a transfer board.

The guideways of the first and/or second number can be releasably connectable to the patient table, i.e. arranged on the patient table with simple or without third device, for example purely manually and using physical strength, and nondestructively detached again. Advantageously, the patient table can be easily adjusted to suit imaging conditions. A versatile use of the patient table is advantageously possible. Alternatively, the guideways of the first and/or second number can also be permanently connected to the patient table.

In an advantageous embodiment of the invention, it is provided that the first axis corresponds to the longitudinal axis of the patient table and the second axis corresponds to the transverse axis of the patient table, such that the transfer board can advantageously be received or transferred back again along the longitudinal axis and along the transverse axis of the patient table.

In a further advantageous embodiment of the invention, the first number of guideways comprises a first plurality of casters which are in contact with the transfer board at the first setting of the relative height, and the second number of guideways comprises a second plurality of casters which are in contact with the transfer board at the second setting of the relative height.

In this embodiment of the invention, the contact surface area between the receiving device and the transfer board accordingly corresponds to the contact surface between the casters of the first or the second number of guideways and the transfer board. The casters can facilitate a steady and uniform movement of the transfer board on the patient table owing to a rolling movement of the casters when the transfer board is displaced, for example by pushing or pulling of the transfer board, in the direction of the rolling movement. As a result, a gentle, shock- and vibration-free reception of the transfer board and of the patient is advantageously possible. Moreover, an easy and low-friction guidance of the transfer board can be achieved as a result.

The casters can in this case have a distinct axis of rotation. In this case the axes of rotation of the first plurality of casters can be arranged perpendicularly to the first axis and the axes of rotation of the second plurality of casters can be arranged perpendicularly to the second axis and consequently present a preferred orientation of the first number of guideways along the first axis and a preferred orientation of the second number of guideways along the second axis. The distinct preferred direction can enable a more reliable displacement and guidance of the transfer board on the patient table. The use of casters without distinct axis of rotation is also possible, however.

In a further advantageous embodiment of the invention, the device for adjusting the relative height additionally comprises a mechanical handle which is embodied in such a way that the relative height is settable mechanically via a mechanical manipulation of the handle.

The handle can be part of a lever device which, when the handle is turned mechanically, leads via a mechanical transmission to a height adjustment of the first and/or the second number of guideways. The turning action can comprise a downward manipulation of the handle in the vertical plane, but also in the horizontal plane, or a lifting and lowering of the handle in the vertical plane. It is also possible for more than one handle to be attached to the receiving device. For example, the receiving device comprises two guideways along the first axis, each of which has a handle for adjusting the relative height. The handle can in this case be operable purely manually, i.e. using physical strength. A simple and time-efficient way of adjusting the relative height is advantageously possible as a result. Advantageously, it is also possible in this way to realize a device for adjusting the relative height which comprises materials that are nonmagnetic and nonconducting and can be used with a magnetic resonance imaging apparatus, for example.

It is furthermore proposed that in a further advantageous embodiment of the invention the device for adjusting the relative height has a guide groove for guiding a guideway of the first number or second number of guideways during the setting of the relative height.

The guide groove can be arranged in the material of the patient table or in a part of the receiving device that is fixed relative to the patient table. A guideway that is adjustable in height can then include an element engaging in the guide groove so that during the setting of the relative height the guideway executes a movement that is securely guided in a vertical plane by the guide groove and that is predefined by the guide groove. A reliable and stable setting of the relative height is advantageously possible as a result. Each height-adjustable guideway can be secured via a guide groove or a guide groove can be assigned to a number of interconnected guideways. In other embodiment variants, the guide groove can also be embodied in the material of the guideway and the patient table or a fixed part of the receiving device can include an element engaging in the guide groove.

An advantageous embodiment of the invention provides that the first number of guideways and/or the second number of guideways are releasably connectable to the patient table.

In particular, the first and/or second number of guideways can be arranged repeatedly on the patient table without a third device, i.e. purely manually, and can also be repeatedly nondestructively detached again from the patient table. For example, the first and/or second number of guideways can be placed onto the patient table. A time-efficient and flexible adaptation of the patient table is advantageously possible by this device. Optional imaging elements, for example, can be more easily arranged on the patient table in this way.

In an advantageous embodiment of the invention, the receiving device and the patient table are embodied in such a way that a receiving space for accommodating a functional imaging element is formed between the receiving device and the patient table.

An optional functional imaging element can be inserted into the receiving space or the patient table and the receiving device can be embodied in such a way that an optional imaging element can be introduced onto the patient table from the side.

The receiving device is embodied for example in such a way that the second number of guideways are releasably connected to the patient table and the functional imaging element can be introduced into the receiving space as soon as the second number or a subset of the second number of guideways are detached from the patient table. After the functional imaging element has been introduced, the second number or a subset of the second number of guideways can be rearranged on the patient table. Other embodiment variants of the receiving device can also be realized in some other form.

The optional functional imaging element is for example a spine coil for a magnetic resonance tomography examination of a patient, the spine coil being arranged underneath the patient and being placed or mounted onto the substantially horizontal surface of the patient table.

Other optional imaging elements may comprise additional electronics, for example. Advantageously, the patient table together with the receiving device can be adapted to suit the imaging conditions.

Furthermore, an advantageous embodiment of the invention provides that the patient table is a patient table of a magnetic resonance tomography imaging apparatus.

In particular, the receiving device in this embodiment substantially comprises a material or materials which is or are nonconducting and nonmagnetic, for example a fiber-reinforced composite material or a plastic material.

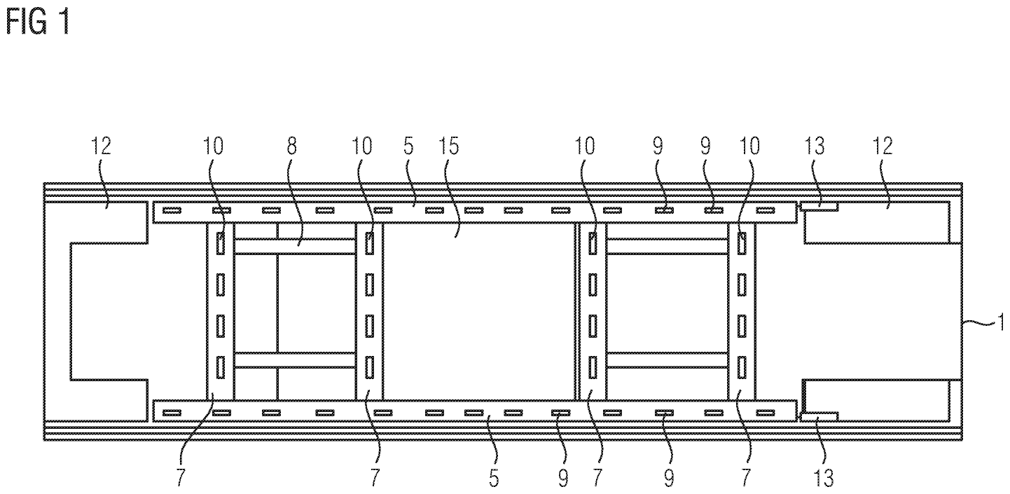

FIG. 1 shows a schematic top plan view onto a patient table (1) according to the invention having a device for receiving a transfer board along two axes that are different from one another, which receiving device has a first number of guideways (5) along the longitudinal axis of the patient table and a second number of guideways (7) along the transverse axis of the patient table (1), some of which are connected via connecting parts (8). In this arrangement, the first number of guideways (5) has a first plurality of casters (9) and the second number of guideways (7) has a second plurality of casters (10). In this embodiment variant, the casters (9,10) each have a distinct axis of rotation and consequently present a preferred orientation for the movement of a transfer board along the longitudinal axis of the patient table (1) as soon as the transfer board comes into contact with the first number of casters (9) and a preferred orientation for the movement of a transfer board along the transverse axis of the patient table (1) as soon as the transfer board comes into contact with the second number of casters (10).

In the embodiment variant shown, the first number of guideways (5) is adjustable in height such that there is a first setting in which the first number of guideways comes into contact with the transfer board via the first plurality of casters (9) and the transfer board can be received along the longitudinal axis of the patient table, and such that there is also a second setting of the relative height in which the second number of guideways (7) comes into contact with the transfer board via the second plurality of casters (10) and the transfer board can be guided along the transverse axis of the patient table (1) while it is being received or transferred back. The contact surface area between the receiving device and the transfer board accordingly corresponds in the first setting to the relative height of the contact surface between the casters (9) of the first number of guideways (5) and the transfer board, and in the second setting to the relative height between the casters (10) of the second number of guideways (7) and the transfer board.

In this arrangement, the device for adjusting the relative height has in each case a handle (13) associated with a guideway of the first number of guideways (5) which are mounted in the region of fixed housing parts (12) of the patient table (1). A lowering or raising of the first number of guideways (5) is made possible with the aid of the handles. The handles (13) can be part of a lever device which, when the handles (13) are turned mechanically, leads via a mechanical transmission to an adjustment in height of the first number of guideways (5). The handles can also be embodied in such a way that they can be raised or lowered in the vertical plane, which leads to a raising or lowering of the guideways (5) either directly or via a transmission. In this case the height of the guideways (5) can be adjustable purely manually, i.e. using physical strength. In other embodiment variants, the handles (13) and/or the device for adjusting the relative height can also be realized in a different way and for example have a motor assistance.

The receiving device for reversibly receiving a transfer board is furthermore embodied in such a way that a functional imaging element (15) can be introduced into a receiving space between the receiving device and the patient table. In this embodiment variant, at least the second number of guideways (7) or else only a subset of the second number of guideways (7) can be detached from the patient table for that purpose and rearranged after the functional imaging element (15) has been introduced. For example, the receiving space comprises a vertical height of 20 to 80 mm, and a width along the transverse axis of the patient table of 200 to 600 mm. In an actual embodiment, the receiving space comprises at least a height of 41.5 mm, a length of 1385 mm and a width of 490 mm. However, the receiving space can also be embodied in some other way.

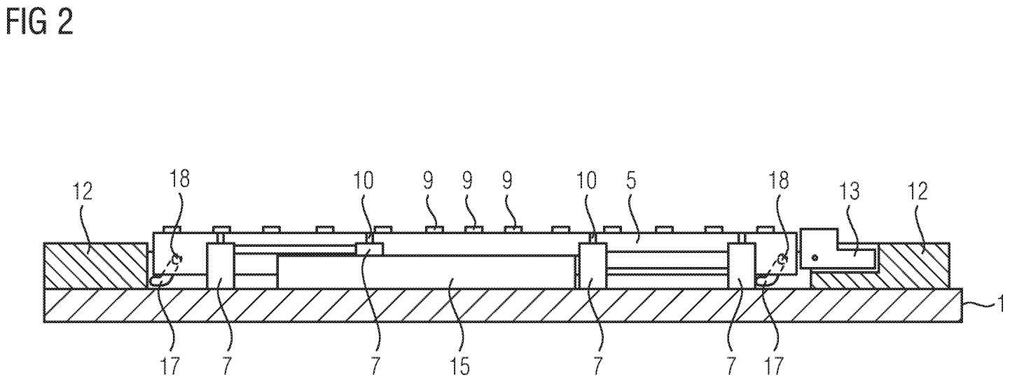

FIG. 2 shows a schematic cross-section through an inventive patient table (1) according to the embodiment variant shown in FIG. 1. The view shows an embodiment of the subset of the second number of guideways (7) having a recess or lower height of the main structure of the guideways, such that the receiving space for the optional functional imaging element (15) is formed between the receiving device and the patient table (1). The second number of guideways (7) in this case bears on the patient table (1) and the subset of the second number of guideways (7) rests on the functional imaging element (15). In other embodiment variants, however, the guideways (7) can also be realized in some other way and do not bear on the patient table and on the functional imaging element (15). The second number of guideways (7) can be releasably connected to the patient table in order to allow the functional imaging element to be introduced.

Also indicated in the schematic cross-section is the course of two guide grooves (17) which are embodied in a fixed part of the patient table (1), for example in a side wall, and which in this view are embodied behind and partially covered by the guideway (5) of the first number of guideways (5). The guideway comprises elements (18) engaging in the guide groove (17) such that during the setting of the relative height the guideway (5) executes a movement that is securely guided in a vertical plane by the guide grooves (17) and that is predefined by the embodiment of the guide grooves (17). The view shows an operating state of the receiving device in which the first number of guideways (5) provide the contact surface area for a transfer board that is to be received and guide the latter along the longitudinal axis of the patient table (1) during its reception.

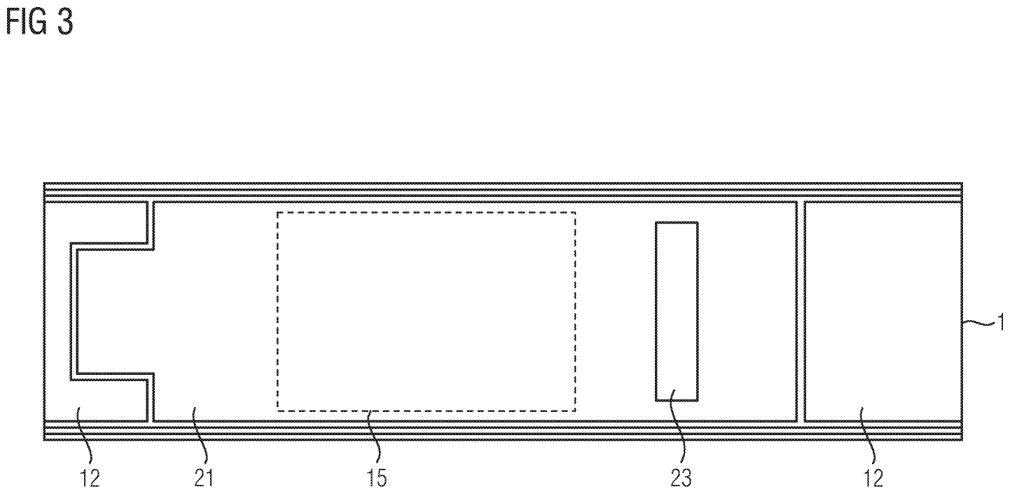

FIG. 3 shows a schematic top plan view onto a patient table (1) according to the invention having a device for receiving a transfer device along two axes that are different from one another, the receiving device comprising a sliding board (21) which is inserted into a recess of the patient table (1) and in combination with surface areas of housing parts (12) of the patient table (1) adjoining the recess forms a receiving surface for the transfer device. In this arrangement, the sliding board (21) fits flush with the housing parts (12) such that it is possible for the transfer device to be received easily and gently in the horizontal plane. In this embodiment variant, the transfer device can be for example a transfer sheet or a transfer board. For example, the transfer board can also have casters on the side facing toward the sliding board (21). The transfer board (21) additionally has a depression (23) for catching and collecting fluid, for example body fluids. In this case the depression is embodied for example to contain at least a volume of 0.5 to 3 liters. In an actual embodiment the depression holds 2 liters of fluid.

The patient table (1) and the sliding board are embodied in such a way that a receiving space for a functional imaging element (15), for example a local coil for a magnetic resonance tomography examination, is formed between the sliding board (21) and the patient table (1). In this example embodiment, the sliding board (21) must be released from the patient table (1) in order to introduce the functional imaging element (15). In other embodiment variants, the patient table (1) can for example have recesses in the sides of the housing through which the functional imaging element (15) can be introduced into the receiving space from the side.

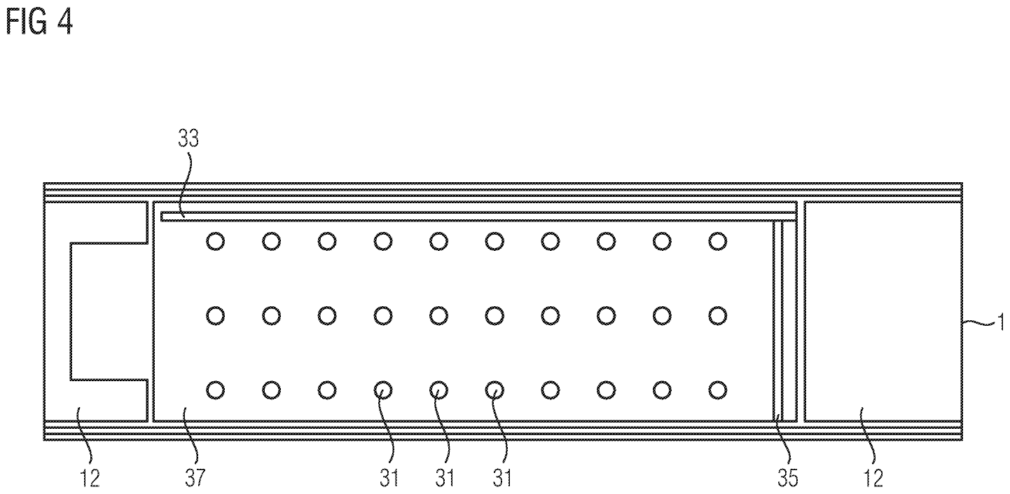

FIG. 4 shows a schematic top plan view onto a patient table (1) according to the invention having a device for receiving a transfer board along two axes that are different from one another by way of ball casters (31) arranged in a horizontal plane. In this embodiment variant, the ball casters (31) are embodied on a level board (37) which is placed on the patient table (1) and which comes into contact with the transfer board when the transfer board is received. The receiving device additionally has a first rail system (33) in the form of a first groove along the longitudinal axis of the patient table and a second rail system (35) in the form of a second groove along the transverse axis of the patient table. The transfer board can then have a corresponding first guide element which can be introduced into the first groove when the transfer board is received along the longitudinal axis and reliably and securely guides the transfer board along the longitudinal axis. The transfer board can then have a corresponding second guide element which can be introduced into the second groove when the transfer board is received along the transverse axis and reliably and securely guides the transfer board along the transverse axis.

Although the invention has been illustrated in greater detail on the basis of the preferred example embodiments, the invention is not limited by the disclosed examples and other variations may be derived herefrom by the person skilled in the art without leaving the scope of protection of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A patient table of an imaging apparatus, comprising:
a receiving device for receiving a transfer board, reversibly transferable to the patient table, the receiving device being arranged on the patient table such that a surface area of the receiving device is in contact with the transfer board when the transfer board is received, and the receiving device being embodied to receive the transfer board reversibly along at least two axes, the at least two axes being different from one another in a horizontal plane.

2. The patient table of claim 1, wherein the receiving device includes a sliding board and wherein the patient table includes a recess into which the sliding board is insertable such that, upon the sliding board being inserted, a surface of the sliding board fits flush, and a surface region of the patient table of the sliding board forming a receiving surface for the transfer board in combination with the surface of the sliding board.

3. The patient table of claim 2, wherein the patient table and the sliding board are embodied such that a receiving space for accommodating a functional imaging element is embodied between the sliding board, upon the sliding board being inserted, and the patient table.

4. The patient table of claim 2, wherein the sliding board includes at least one depression, embodied to catch fluid.

5. The patient table of claim 1, wherein
the receiving device includes a plurality of ball casters in a horizontal plane, in contact with a transfer board upon the transfer board being received,
the receiving device includes a first rail system along a first axis and a second rail system along a second axis, the second axis being different from the first axis, and
the first rail system is configured to guide the transfer board along the first axis when the transfer board is being received, and the second rail system is configured to guide the transfer board along the second axis when the transfer board is being received.

6. The patient table of claim 1, wherein the receiving device includes
a first number of guideways to guide a transfer board along a first axis,
a second number of guideways to guide the transfer board along a second axis, the second axis being different from the first axis, and
a device for adjusting a relative height between the first number of guideways and the second number of guideways, wherein a first setting of the relative height is usable to configure the first number of guideways to guide the transfer board and wherein a second setting of the relative height is usable to configure the second number of guideways to guide the transfer board.

7. The patient table of claim 6, wherein the first axis corresponds to a longitudinal axis of the patient table and the second axis corresponds to a transverse axis of the patient table.

8. The patient table of claim 6, wherein the first number of guideways include a first plurality of casters, configured to be in contact with the transfer board at the first setting of the relative height and wherein the second number of guideways includes a second plurality of casters, configured to be in contact with the transfer board at the second setting of the relative height.

9. The patient table of claim 6, wherein the device for adjusting the relative height includes a mechanical handle, embodied such the relative height is settable via a mechanical manipulation of the mechanical handle.

10. The patient table of claim 6, wherein the device for adjusting the relative height includes a guide groove for guiding a guideway of the first number of guideways or second number of guideways during setting of the relative height.

11. The patient table of claim 6, wherein at least one of the first number of guideways and the second number of guideways is releasably connectable to the patient table.

12. The patient table of claim 6, wherein the receiving device and the patient table are embodied such that a receiving space for accommodating a functional imaging element is formed between the receiving device and the patient table.

13. A magnetic resonance tomography imaging apparatus, comprising:
the patient table of claim 1.

14. The patient table of claim 3, wherein the sliding board includes at least one depression, embodied to catch fluid.

15. The patient table of claim 2, wherein
the receiving device includes a plurality of ball casters in a horizontal plane, in contact with a transfer board upon the transfer board being received,
the receiving device includes a first rail system along a first axis and a second rail system along a second axis, the second axis being different from the first axis, and
the first rail system is configured to guide the transfer board along the first axis when the transfer board is being received, and the second rail system is configured to guide the transfer board along the second axis when the transfer board is being received.

16. The patient table of claim 1, wherein the receiving device includes
a first number of guideways to guide a transfer board along a first axis, the first number of guideways including a first plurality of casters, configured to be in contact with the transfer board at the first setting of the relative height,
a second number of guideways to guide the transfer board along a second axis, the second axis being different from the first axis, the second number of guideways including a second plurality of casters, configured to be in contact with the transfer board at the second setting of the relative height, and
a device for adjusting a relative height between the first number of guideways and the second number of guideways, the device including a mechanical handle, embodied such the relative height is settable via a mechanical manipulation of the mechanical handle.

17. The patient table of claim 1, wherein the receiving device includes
a first number of guideways to guide a transfer board along a first axis, the first number of guideways including a first plurality of casters, configured to be in contact with the transfer board at the first setting of the relative height,
a second number of guideways to guide the transfer board along a second axis, the second axis being different from the first axis, the second number of guideways including a second plurality of casters, configured to be in contact with the transfer board at the second setting of the relative height, and
a device for adjusting a relative height between the first number of guideways and the second number of guideways, the device including a mechanical handle, embodied such the relative height is settable via a mechanical manipulation of the mechanical handle, and
wherein at least one of the first number of guideways and the second number of guideways is releasably connectable to the patient table.

18. The patient table of claim 1, wherein the receiving device includes
a first number of guideways to guide a transfer board along a first axis, the first number of guideways including a first plurality of casters, configured to be in contact with the transfer board at the first setting of the relative height,
a second number of guideways to guide the transfer board along a second axis, the second axis being different from the first axis, the second number of guideways including a second plurality of casters, configured to be in contact with the transfer board at the second setting of the relative height, and
a device for adjusting a relative height between the first number of guideways and the second number of guideways, and
wherein at least one of the first number of guideways and the second number of guideways is releasably connectable to the patient table.

19. The patient table of claim 1, wherein the receiving device includes
a first number of guideways to guide a transfer board along a first axis, the first number of guideways including a first plurality of casters, configured to be in contact with the transfer board at the first setting of the relative height,
a second number of guideways to guide the transfer board along a second axis, the second axis being different from the first axis, the second number of guideways including a second plurality of casters, configured to be in contact with the transfer board at the second setting of the relative height, and
a device for adjusting a relative height between the first number of guideways and the second number of guideways,
wherein at least one of the first number of guideways and the second number of guideways is releasably connectable to the patient table, and
wherein the device for adjusting the relative height includes a guide groove for guiding a guideway of the first number of guideways or second number of guideways during setting of the relative height.

20. The patient table of claim 9, wherein the device for adjusting the relative height includes a guide groove for guiding a guideway of the first number of guideways or second number of guideways during setting of the relative height.
